(19) United States
(12) Patent Application Publication
(10) Pub. No.: US 2005/0124971 A1
(43) Pub. Date: Jun. 9, 2005

(54) Methods and devices for catheter-based intracoronary myocardial delivery of cellular, genetic or biological materials

(75) Inventors: Walter J. Koch, Durham, NC (US); Sitaram M. Emani, Durham, NC (US); Donald D. Glower, Durham, NC (US); Ashish S. Shah, Durham, NC (US)

Correspondence Address: NIXON & VANDERHYE, PC, 1100 N GLEBE ROAD, 8TH FLOOR, ARLINGTON, VA, 22201-4714, US

(73) Assignee: Duke University, Durham, NC (US)

(21) Appl. No.: 10/895,261

(22) Filed: Jul. 21, 2004

Related U.S. Application Data
(60) Provisional application No. 60/488,443, filed on Jul. 21, 2003.

(51) Int. Cl. A61M 31/00

(52) U.S. Cl. 604/509

(57) ABSTRACT

Methods and systems infuse therapeutic materials into a vascular vessel by means of a catheter based infusion system. In especially preferred forms, the infusate (which comprises a therapeutic material) is infused through a distal end of the catheter and into the vascular vessel by delivering the infusate to the catheter at a substantially constant flow rate while simultaneously imparting a pressure amplitude and frequency to the infusate in dependence upon a sensed pressure condition within the vessel. Preferably, and the pressure amplitude imparted to the constant flow rate of infusate is about twice the vessel systolic pressure. The present invention is therefore especially well suited for the catheter-based infusion of relatively large particles, such as cellular, genetic, viral, polymeric or proteinaceous materials, intravascularly in dependence upon the pressure and flow characteristics of the infusate.

### CROSS-REFERENCE TO RELATED APPLICATION

This application is based on, and claims domestic priority benefits under 35 USC §119(e) from, U.S. Provisional Patent Application Ser. No. 60/488,443 filed on Jul. 21, 2003, the entire content of which is expressly incorporated hereinto by reference.

### GOVERNMENT RIGHTS

This invention was made with Government support under Grant Nos. HL59533 and HL56205 awarded by the National Institute of Health. The Government has certain rights in the invention.

### FIELD OF THE INVENTION

The present invention relates generally to methods and devices for the delivery of cellular, genetic or biological materials. In especially preferred forms, the present invention is embodied in methods and systems to facilitate adenoviral-mediated gene transfer to the mycocardium utilizing subselective coronary catheterization.

### BACKGROUND AND SUMMARY OF THE INVENTION

Numerous devices exist currently for the administration of intravascular medications to patients that utilize a constant flow rate infusion pump coupled to an intravenous or intraarterial catheter. These devices, both extracorporeal and implantable, simply deliver the medication into the intravascular space and rely upon transcapillary diffusion to attain therapeutic tissue levels. Recently, novel means of tissue therapeutics have included the delivery of larger cellular, viral, genetic or polymeric materials to damaged tissues. Most investigators have utilized direct tissue injection as the means of therapy, as it ensures local delivery. Intravascular delivery of these larger therapeutic particles has resulted in very low tissue penetration and therapeutic failure.

Recently, several investigators, including the present inventors, have described success with intra-arterial delivery of an adenovirus containing DNA to the heart. See in this regard, Hajjar et al., “Modulation of ventricular function through gene transfer in vivo”, Proc Natl Acad Sci USA 95:5251-5256 (1998), Maurice et al., “Enhancement of cardiac function after adenoviral-mediated in vivo intracoronary β₂-adrenergic receptor gene delivery”, J Clin Invest 104:21-29 (1999) and Schmidt et al., “Restoration of diastolic function in senescent rat hearts through adenoriral gene transfer of sarcoplasmic reticulum Ca²⁺-ATPase”, Circulation 101:790-796 (2000).¹ These investigators were able to obtain tissue transgene expression by crossclamping the aorta and delivering the virus into the proximal aorta, thereby utilizing the pulsatile driving pressure of the heart to augment local tissue penetration by the virus.
¹ The entire content of these publications and each publication cited below is expressly incorporated hereinto by reference.

The results of prior investigators have been reproduced by the present inventors utilizing a percutaneous approach. More specifically, the present inventors showed reproducible myocardial transgene expression following infusion of an adenoviral vector into the coronary circulation at high flow and pressure after catheterization of a single coronary artery. Shah et al., “Intracoronary adenovirus-mediated delivery and overexpression of the β₂-adrenergic receptor in the heart: prospects for molecular ventricular assistance”, Circulation 101:408-414 (2000). This included percutaneous sub-selective catheterization and adenoviral-mediated gene delivery to the left ventricle of a failing heart. Shah et al., “In vivo ventricular gene delivery of a β-adrenergic receptor kinase inhibitor to the failing heart reverses cardiac dysfunction”, Circulation 103:1311-1316 (2001).

Devices have been proposed in the past for use during cardiac bypass, intra-aortic balloon pumping (U.S. Pat. No. 4,493,697), and perfusion of isolated donor organs to prevent ischemic injury prior to transplantation. Such prior devices are, however, typically designed to perfuse the capillary bed of interest and metabolically support the tissue for a limited duration. In this regard, the conventional devices typically utilize pulsatile energy to accomplish tissue perfusion, yet differ significantly in the therapeutic goal and means of generating the pulsatile energy.

Cardioplegia catheters (U.S. Pat. No. 5,913,842) utilized in cardiopulmonary bypass allow cardioplegia solution to be delivered to the coronary circulation at a desired flow rate, but do not allow the operator to program a desired intraluminal pressure or superimposed waveform. The intraaortic balloon pump delivers pulsatile energy into the vascular system by sequential inflation and deflation of the intraaortic balloon. Although such a system allows for intermittent injection of intravascular medication, the pressure and flow characteristics of the infusate are not controllable.

Devices are also known which are capable of dissolving intravascular thrombi by passing a catheter into the core of a thrombus and delivering thrombolytic drugs directly into the clot in a pulsatile manner. Such devices differ from the present invention in that the thrombolytic catheter contains multiple side holes with an occluded distal end, thereby allowing pulsed fluid to extrude circumferentially from the catheter. In such a conventional device, therefore, the operator does not specify the desired pressure or flow waveform, but instead the pulses are manually generated.

There exists in this art, therefore, a definite need for catheter-based methods and devices that would allow successful tissue delivery of large particles, such as cellular, genetic, viral, polymeric or proteinaceous materials intravascularly in dependence upon the pressure and flow characteristics of the infusate. It is towards fulfilling such a need that the present invention is directed.

Broadly, the present invention is embodied in methods and devices for performing cellular, genetic, viral, biologic, or molecular based therapy. More particularly, the present invention is embodied in catheter-based methods and systems for delivery of therapeutic materials to tissues. In especially preferred forms, the present invention is embodied in methods and devices capable of controlling intravascular pressure and flow conditions as a means of facilitating the delivery of novel therapeutic materials.

The methods and systems of the present invention therefore are especially adapted to infuse therapeutic materials into a vascular vessel by means of a catheter based infusion system. In especially preferred forms, the infusate (which comprises a therapeutic material) is infused through a distal end of the catheter and into the vascular vessel by delivering the infusate to the catheter at a substantially constant flow rate while simultaneously imparting a pressure amplitude and frequency to the infusate in dependence upon a sensed pressure condition within the vessel. Preferably, and the pressure amplitude imparted to the constant flow rate of infusate is about twice the vessel systolic pressure. The present invention is therefore especially well suited for the catheter-based infusion of relatively large particles, such as cellular, genetic, viral, polymeric or proteinaceous materials, intravascularly in dependence upon the pressure and flow characteristics of the infusate.

These and other aspects and advantages will become more apparent after careful consideration is given to the following detailed description of the preferred exemplary embodiments thereof.

### BRIEF DESCRIPTION OF THE ACCOMPANYING DRAWINGS

Reference will hereinafter be made to the accompanying drawings, wherein

FIG. 1A is a schematic diagram representing the preferred hardware that may be employed to control the catheter system of the present invention;

FIG. 1B is a schematic view of a catheter system for the controlled in vivo delivery of cellular, genetic or biological material to myocardium in accordance with the present invention; and

FIG. 2 is a schematic diagram representing a preferred computer control logic for the catheter system of this invention.

### DETAILED DESCRIPTION OF THE INVENTION

One principal aspect of the present invention is the discovery that successful tissue delivery of large particles such as cellular, genetic, viral, polymeric, or proteinaceous materials depends upon the pressure and flow characteristics of the infusate. Although the exact mechanism is unclear at this time, it is believed that disruption of the basement membrane may be required to allow larger materials to exit the capillary bed and attain therapeutic tissue levels. The present invention is therefore embodied in catheter-based methods and devices driven by a pulsatile infusion pump that allows the operator to essentially “design” the desired pressure or flow waveform characteristic of infusate delivery.

No presently known device has been capable of infusing therapeutic materials into a vascular lumen under pressure or flow conditions as specified by an operator. As genetic, cellular, and novel therapeutic modalities approach clinical use, intravascular delivery will provide an attractive alternative to direct tissue injection. The advantages of an intravascular delivery may include homogenous tissue delivery and the ability to perform the procedure through a minimally invasive or percutaneous approach. The rare success heretofore with direct intravascular delivery of large therapeutic products may be attributed to the lack of appropriate pressure or flow conditions. The current invention will facilitate genetic therapy via intravascular delivery, which has previously been largely unsuccessful.

Various disease states are associated with aberrations in cellular, genetic or molecular composition. A novel therapeutic approach to these disease states involves the introduction of cellular, genetic, or biologic material into tissue so as to ameliorate the morbidity and mortality consequent to the diseased state. Novel therapeutics includes the use of cellular, genetic, viral, or biologic products to treat pathologic tissue (acquired or congenital) that contributes to a disease process. “Gene therapy”, or the use of genetic material (DNA or RNA) to ameliorate a disease process, is an example of a novel therapeutic modality that is gaining clinical utility. For example, investigators have recently reported success with local injections of a growth factor (VEGF) into the leg of patients with arterial insufficiency from vascular disease. Direct intravascular delivery of this large protein may provide advantages over direct tissue injection. The present invention therefore finds particular utility to facilitate such therapy.

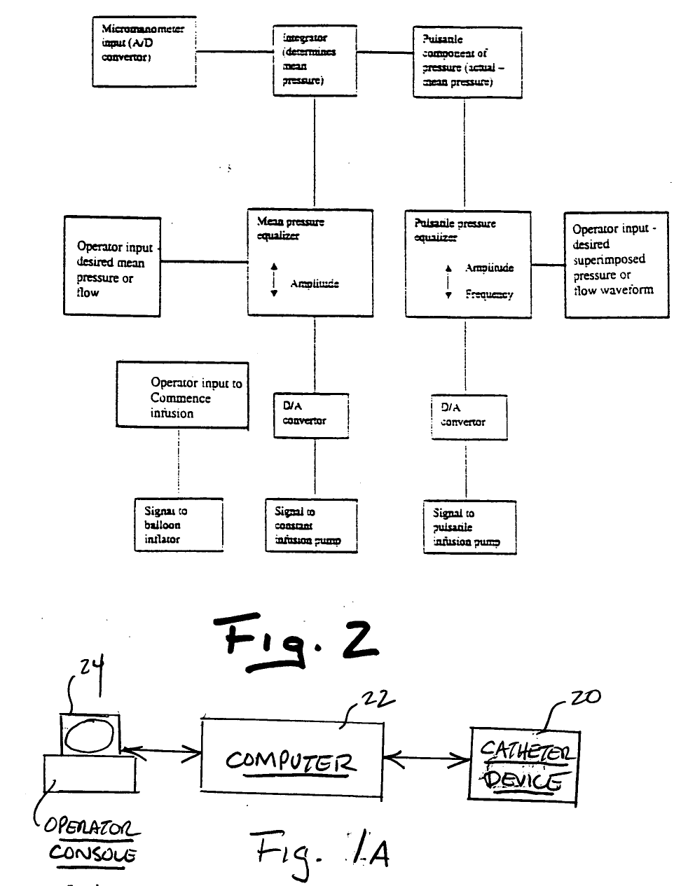

In one presently preferred embodiment of the system as shown in FIG. 1A, a computer 22 serves as the interface between the operator via console 24 and the catheter device 20.

One presently preferred embodiment of a catheter-based system 20 in accordance with the present invention is illustrated schematically in FIG. 1B. As shown, the device contains a flexible hollow lumen catheter (1) with an inflatable low-pressure balloon (3) at the distal end. The balloon (3) prevents retrograde flow of infusate during the procedure, but has a low elastic modulus to prevent vascular injury at the time of inflation. A micromanometer (2) distal to the balloon measures intravascular pressure during infusion. The diameters of the catheter and balloon are not critical and can in fact vary depending upon the size of the target vessel.

The proximal end of the catheter (1) contains a port (not shown) to which a syringe can be attached and through which intravascular contrast may be injected. The catheter (1) is coupled to a loading chamber (4), into which the infusate is placed prior to infusion. The loading chamber (4) maintains conditions appropriate for storage of infusate, for example, temperature, pH, electrolyte content, agitation to prevent precipitation, and like conditions. The loading chamber (4) is coupled operatively to a variable constant rate infusion pump (10) and a pulsatile waveform generator (7). The constant and pulsatile infusion components contain a driving fluid with an electrolyte and pH content appropriate to ensure infusate stability.

The constant infusion pump (10) includes a movable piston that infuses fluid into the loading chamber (4) and anterograde into the catheter. The infusion pump (10) is capable of delivering high flow rates needed to attain adequate intravascular mean pressure.

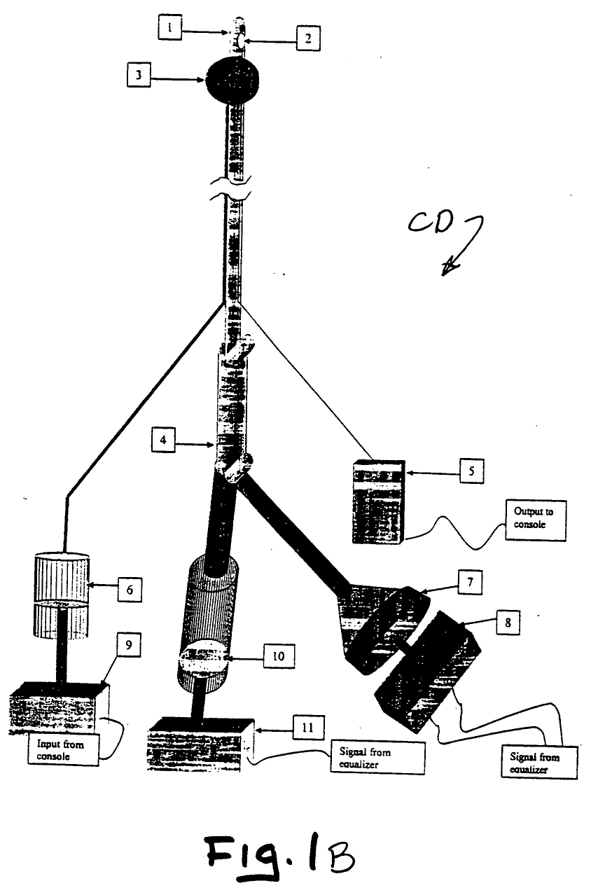

The rate of infusion is controlled by the piston actuator (11), which in turn receives input from the console (see FIGS. 1A and 2). The signal amplitude controls the rate of piston advancement, and thus the flow rate. Preferably, a rotating screw (not shown) is provided upon which the piston driver (11) is slidably mounted so as to drive the constant infusion pump (10). As the screw rotates, translational motion of the piston (10) expels saline from the piston housing into the loading chamber (4) thereby displacing the infusate anterograde into the arterial lumen.

The pulsatile waveform generator (7) includes a diaphragm (not shown) enclosed within a housing. The diaphragm is connected to a piston, which is driven by an oscillatory motor (8). Fluid within the housing, in continuity with the infusate, is oscillated by the to-and-fro motion of the diaphragm. The amplitude and frequency of the oscillations are determined by the amplitude and frequency of the signal from the console (24) (see FIG. 1A).

Inflation of the low-pressure balloon is accomplished by infusion of a fixed volume of fluid (e.g., 0.9% saline) from the balloon inflator (6). Input from the console determines the timing and rate of inflation, as well as the total balloon volume (9). Balloon inflation precedes infusion of therapeutic materials, and this timing is controlled by the signal from the console (24). The micromanometer (2) is connected to a calibration box (5), and signal output is sent to the console (24) for continuous monitoring.

In use, with reference to FIG. 2, the operator will place the catheter tip into the artery or vein of choice, choose the desired mean and superimposed pressure waveform, and load the sample into the loading chamber. The amplitude and frequency of the superimposed waveform are determined by the operator and will vary depending upon the specific vessel to be catheterized. In this regard, it has been found that the pressure amplitude is most preferably about twice systolic pressure within the vessel.

Upon actuation, the computer (24) will activate the piston driver (9) and begin filling the occlusion balloon (3) with normal saline to the recommended volume. Inflation of the occlusion balloon (3) prevents retrograde escape of infusate around the catheter. The flow rate of infusate from the loading chamber (4) is incrementally increased by means of the piston actuator (11) and the constant rate infusion pump (10) until the mean pressure in the vessel as measured by the distal transducer (2) reaches the desired mean pressure. Simultaneously, the computer activates the diaphragm of the function generator (8) according to the desired settings. The amplitude and frequency of diaphragmatic displacement can be modified according to the real-time intravascular pressure measurement.

The computer monitors input from the transducer and appropriate modifications to the constant flow rate will be implemented to maintain the desired mean pressure. Input from the micromanometer is converted into digital signal via A/D converter (FIG. 2). Instantaneous mean pressure

$${\int }_{BP1}^{BP2} P ( t ) ⅆ t / T$$

is calculated as the time averaged signal by the integrator. The mean pressure for a single pulse is calculated as:
where BP1 is the beginning of the pulse, BP2 is the beginning of the following pulse, and T is the time between BP1 and BP2. The beginning of a pulse is defined as the time point in the cycle where dP/dt is maximal as shown in the waveform below:

[image]

The mean pressure equalizer compares actual and desired mean pressure, and modifies signal amplitude. If (desired)−(actual) pressure>0, then the amplitude is increased. If it is <0, then the amplitude is decreased. The time to steady state pressure will be minimized by the beat to beat analysis.

To avoid swings in amplitude resulting from the delayed pressure response to changes in flow rate, several modifications may be made in the integrator software to allow for multiple beat analysis, or analysis of pulse beats arriving several milliseconds following change in constant flow rate. Output from the mean pressure equalizer is processed by a D/A converter, and sent to the constant infusion pump for implementation.

The pulsatile component of the measured pressure is determined by subtracting the mean pressure from the actual pressure. The waveform that results has an amplitude and frequency that are compared to the input specified by the operator. The type of waveform (sinusoidal, square, exponential, crescendo etc.) specified by the operator is compared to the actual waveform. The pulsatile pressure equalizer modifies the amplitude, frequency, and shape of the signal as needed to generate the desired pressure waveform. Signal from the pulsatile pressure equalizer is processed by a D/A converter and sent to the pulsatile infusion pump where diaphragmatic oscillations generate the desired pressure waveform.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A method of the intravascular infusion of therapeutic materials comprising the steps of:
(i) introducing an infusion catheter into a vascular vessel;
(ii) providing an infusate which comprises a therapeutic material;
(iii) infusing the infusate through a distal end of the catheter and into the vascular vessel by controlling pressure and flow rate characteristics of the infusate so as to correspond to a desired mean pressure condition within the vascular vessel.

2. The method of claim 1, wherein step (iii) comprises delivering the infusate at a substantially constant flow rate to the catheter while simultaneously imparting a pulsatile pressure amplitude and frequency condition thereto.

3. The method of claim 1, wherein step (iii) is practiced by determining a systolic pressure condition with in the vascular vessel, and delivering the infusate at a flow rate which is about twice a vascular flow rate within the vessel at said systolic pressure condition.

4. The method of claim 1, which comprises preventing retrograde flow of the infusate into the distal end of the catheter.

5. The method of claim 4, wherein said step of preventing retrograde infusate flow comprises providing an inflatable balloon near the distal end of the catheter, and inflating the balloon prior to step (iii).

6. A system for the intravascular infusion of therapeutic materials comprising the steps of:
an infusion catheter sized and configured to be introduced into a vascular vessel;
a chamber adapted to provide a source of a therapeutic material-containing infusate;
an infusion system operatively connecting the chamber and the infusion catheter for infusing the infusate through a distal end of the catheter and into the vascular vessel, said infusion system comprising a controller for controlling pressure and flow rate characteristics of the infusate so as to correspond to a desired mean pressure condition within the vascular vessel.

7. The system of claim 6, wherein the catheter includes a pressure sensor for sensing the desired mean pressure condition within the vascular vessel.

8. The system of claim 6, wherein the controller comprises means for delivering the infusate at a substantially constant flow rate to the catheter while simultaneously imparting a pulsatile pressure condition thereto.

9. The system of claim 6, wherein the controller delivers the infusate at a flow rate which is about twice a vascular flow rate within the vessel at said systolic pressure condition.

10. The system of claim 6, which comprises an inflatable balloon at the distal end of the catheter for preventing retrograde flow of the infusate.

11. A method for the intravascular infusion of an infusate comprising the steps of:
(i) introducing an infusion catheter into a vascular vessel;
(ii) providing an infusate which comprises a therapeutic material;
(iii) sensing a pressure condition within the vascular vessel; and
(iv) infusing the infusate through a distal end of the catheter and into the vascular vessel by delivering the infusate to the catheter at a substantially constant flow rate while simultaneously imparting a pressure amplitude and frequency to the infusate in dependence upon the sensed pressure condition within the vessel.

12. The method of claim 11, wherein step (iv) is practiced so that the pressure amplitude imparted to the infusate is about twice systolic pressure within the vessel.

13. The method of claim 11, which further comprises preventing retrograde flow of the infusate into the distal end of the catheter.

14. The method of claim 13, wherein said step of preventing retrograde infusate flow comprises providing an inflatable balloon near the distal end of the catheter, and inflating the balloon prior to step (iv).

15. A system for the intravascular infusion of therapeutic materials comprising the steps of:
an infusion catheter sized and configured to be introduced into a vascular vessel;
a chamber adapted to provide a source of a therapeutic material-containing infusate; and
an infusion system operatively connecting the chamber and the infusion catheter for infusing the infusate through a distal end of the catheter and into the vascular vessel, wherein
said infusion system comprises a controller for controllably delivering the infusate to the catheter at a substantially constant flow rate while simultaneously imparting a pressure amplitude and frequency to the infusate in dependence upon the sensed pressure condition within the vessel

16. The system of claim 15, wherein said controller controllably delivers the infusate to the catheter at a pressure amplitude which is about twice systolic pressure within the vessel.

17. The system of claim 15, wherein the catheter includes a pressure sensor for sensing a desired mean pressure condition within the vascular vessel, and wherein said controller operates to impart a pressure amplitude and frequency to the infusate in dependence upon said sensed mean pressure condition.

18. The system of claim 15, which comprises an inflatable balloon at the distal end of the catheter for preventing retrograde flow of the infusate.
